(19) United States
(12) Patent Application Publication
(10) Pub. No.: US 2023/0084699 A1
(43) Pub. Date: Mar. 16, 2023

(54) METHODS FOR FORMING DIRECTIONAL MYCELIUM FIBERS

(71) Applicant: EMERGY INC., Boulder, CO (US)

(72) Inventors: Justin WHITELEY, Lafayette, CO (US); Mitchell Tyler HUGGINS, Boulder, CO (US)

(73) Assignee: EMERGY INC., Boulder, CO (US)

(21) Appl. No.: 17/904,217

(22) PCT Filed: Feb. 10, 2021
(86) PCT No.: PCT/US2021/017494

§ 371 Date: Aug. 12, 2022

Related U.S. Application Data
(60) Provisional application No. 62/976,957, filed on Feb. 14, 2020.

(51) Int. Cl. A01G 18/10; A23J 3/20; A23J 3/22; A23P 30/10; A01G 18/20

(52) U.S. Cl. CPC A01G 18/10; A23J 3/20; A23J 3/227; A23P 30/10; A01G 18/20

(57) ABSTRACT

A method of forming an edible meat substitute product includes growing fungal cells in a growth media such that the fungal cells produce a mycelium mass having a protein content of greater than 40 wt % of a dry mass of the mycelium mass. The method includes separating the mycelium mass from the growth media. The method includes disposing the mycelium mass on a base of a mold. The method includes applying a uniaxial pressure to the mycelium mass via a follower to produce a compacted mycelium mass having a moisture content in a range of 65 vol % to 85 vol % and having a shape corresponding to a shape of the mold. A plurality of fibers of the compacted mycelium mass are aligned in a direction orthogonal to the direction of the applied uniaxial pressure.

### CROSS-REFERENCE TO RELATED PATENT APPLICATIONS

The present application claims the benefit of and priority to U.S. Provisional Patent Application No. 62/976,957 filed Feb. 14, 2020, the entire disclosure of which is incorporated by reference herein.

### TECHNICAL FIELD

The present disclosure relates generally to the field of fungal mycelium based edible meat substitute products.

### BACKGROUND

Demand for edible products that can provide a high protein content which is drawn from a non-animal source is increasing. Driven by increasing awareness of personal health, edible products that include non-animal sourced components such as proteins and fibers are considered as a healthier alternative to animal protein based products. In particular, there is growing demand for edible meat substitutes that mimic meat in its composition and texture but are composed of non-animal components, which can reduce reliance on animals such as cows, chicken, and pigs, and reduce the carbon footprint posed by such animals. Thus, there is a need for non-animal protein sources that can facilitate large scale production and adoption of non-animal based edible products.

### SUMMARY

Embodiments described herein relate generally to methods for forming directional mycelium fibers for obtaining edible meat substitute products that resemble animal meat in their texture and morphology.

In some embodiments, a method of forming an edible meat substitute product comprises growing fungal cells in a growth media such that the fungal cells produce a mycelium mass having a protein content of greater than 40 wt % of a dry mass of the mycelium mass; separating the mycelium mass from the growth media; disposing the mycelium mass on a base of a mold, the mold having sidewalls extending from the base, at least the base of the mold being perforated; and applying a uniaxial pressure to the mycelium mass via a follower to produce a compacted mycelium mass having a moisture content between 65 vol % and 85 vol % and having a shape corresponding to a shape of the mold, wherein a plurality of fibers of the compacted mycelium mass are aligned in a direction orthogonal to the direction of the applied uniaxial pressure. Reorienting the compacted mycelium in different planes and making cuts or slices can result in varying hardness and toughness values that can correspond to different types of animal meat such as chicken and beef.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is a flow chart of an example method for forming directional mycelium fibers, according to an embodiment.

FIG. 2A is a perspective view of a mycelium block obtained by the method of FIG. 1.

FIGS. 2B-2D illustrate cross-sectional views of the mycelium block of FIG. 2A taken along different planes.

FIG. 3A is a perspective view of a mold with a perforated base, according to an embodiment.

FIG. 3B illustrates a perspective view of a mold with a perforated base and perforated sidewalls, according to another embodiment.

FIG. 4A illustrates a plot of hardness values for compacted mycelium that has been cut in different orientations and correspond to different cuts of meat, according to another embodiment.

FIG. 4B illustrates a plot of toughness values for compacted mycelium that has been cut in different orientations and correspond to different cuts of meat, according to another embodiment.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate generally to methods for forming directional mycelium fibers for obtaining edible meat substitute products that resemble animal meat in their texture and morphology. Particularly, various embodiments described herein provide methods of growing fungal cells to produce a mycelium mass, separating the mycelium mass, disposing the mycelium mass on a base of a mold, and applying a uniaxial pressure to the mycelium mass via a follower to produce a compacted mycelium mass. Applying the uniaxial pressure to the mycelium mass produces long-range fibers in the plane orthogonal to the force. A plurality of fibers of the compacted mycelium mass can be aligned in a direction orthogonal to the direction of the applied uniaxial pressure. Textural changes can be achieved by slicing compacted mycelium mass along a predetermined plane. Various embodiments also relate to adding food additives to form an edible food product or edible meat substitute product. The edible meat substitute product can include a mycelium mass having a protein content of greater than 40 wt % of a dry mass of the mycelium mass.

Various embodiments of the methods of growing fungal mycelium and forming edible products therefrom may provide one or more benefits including, for example: (1) providing edible products that include protein from a non-animal source, i.e., fungal mycelium, thereby reducing dependence on animal sources of proteins and reducing their carbon footprint; and (2) providing edible meat substitute products that feel and taste like real meat while delivering a high protein content.

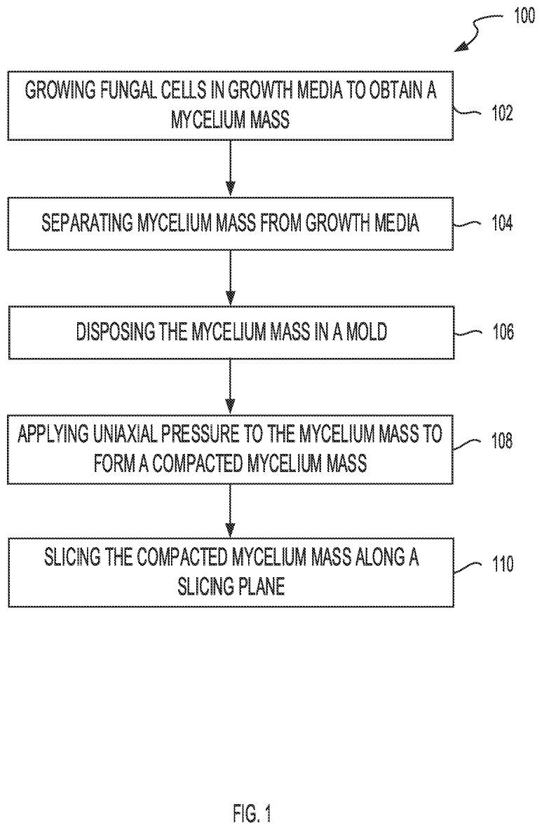

FIG. 1 illustrates a block diagram of an example method 100 for forming an edible meat substitute product, according to an embodiment. In brief overview, the method 100 may include growing fungal cells in a growth media, at 102. The method 100 may include separating mycelium mass from the growth media, at 104. The method 100 may include disposing the mycelium mass in a mold, at 106. The method 100 may include applying uniaxial pressure to the mycelium mass to form a compacted mycelium mass, at 108. The method 100 may include slicing the compacted mycelium mass along a slicing plane, at 110.

In further detail, the method 100 may include growing fungal cells in a growth media, at 102. The fungal cells can include fungi from Ascomycota and Zygomycota, including the genera Aspergillus, Fusarium, Neurospora, and Monascus. Other species include edible varieties of Basidiomycota and genera Lentinula. One genus is Neurospora, which is used in food production through solid fermentation. The genus of Neurospora are known for highly efficient biomass production as well as ability to break down complex carbohydrates. For certain species of Neurospora, no known allergies have been detected and no levels of mycotoxins are produced. In addition to monocultures of filamentous fungi, multiple strains can be cultivated at once to tune the protein, amino acid, mineral, texture, and flavor profiles of the final biomass.

The growth media may be contained in a vessel, such as a vat capable of growing several kilograms of the fungal mycelium. The growth media can be referred to as an original growth media. The method 100 may include growing fungal cells in a growth media such that the fungal cells produce mycelium. The growth media can include nutrients (e.g., sugar, nitrogen-containing compounds, or phosphate-containing compounds). The growth media can include one or more of a sugar, a nitrogen-containing compound, and a phosphate-containing compound. The sugar can be in the range of 5 g/L to 50 g/L. For example, the sugar can be 5 g/L, 10 g/L, 20 g/L, 30 g/L, 40 g/L, or 50 g/L, inclusive. The sugar can include sucrose, glucose, fructose, molasses, or a mixture of sugars. The nitrogen-containing compound can be in the range of 0.5 g/L to 10 g/L. For example, the nitrogen-containing compound can be 0.5 g/L, 1 g/L, 2 g/L, 3 g/L, 4 g/L, 5 g/L, or 10 g/L, inclusive. The nitrogen-containing compound can include ammonium hydroxide, ammonium nitrate, ammonium sulfate, ammonium chloride, urea, yeast extract, peptone, or a mixture of nitrogen-containing compounds. The phosphate-containing compound can be in the range of 0.1 g/L to 5 g/L. For example, the phosphate-containing compound can be 0.1 g/L, 0.2 g/L, 0.3 g/L, 0.4 g/L, 0.5 g/L, 1 g/L, 2 g/L, 3 g/L, 4 g/L, or 5 g/L, inclusive. The phosphate-containing compound can be potassium phosphate, sodium phosphate, phosphoric acid, or a mixture of phosphate-containing compounds.

The fungal cells can be grown at a temperature in a range of 25° C. to 40° C., inclusive. The fungal cells can be grown in a range of 12 hours to 48 hours, inclusive. Growing fungal cells can produce a yield of 5 g/L to 20 g/L of fungal cell dry weight. The mycelium can have a protein content of greater than 40 wt % (dry weight). In some embodiments, the mycelium may have a protein content of 50% to 65%, inclusive (dry weight). The mycelium can have a combined methionine and cysteine content of at least 25 mg/g crude protein.

In some embodiments, the method 100 may include removing a volume of a broth (e.g., siphoned broth). The siphoned broth can contain the fungal cells and the growth media. For example, the siphoned broth can include a solution containing the fungal cells and the growth media. Removing a volume of broth can include discretely removing a volume of broth. For example, a volume of broth can be siphoned from a container containing the broth in a batch process, or be continuously removed from the broth. For example, a volume of broth can flow out of the container containing the broth in a continuous process.

The method 100 may include adding fresh growth media to a container containing the broth. The broth can be a fermentation broth. Nutrients (e.g., sugar, phosphate-containing compound, or nitrogen-containing compound) can be added in a batch growth configuration. For example, the nutrients can be added after a predetermined amount of time (e.g., after 1 hour, 2 hours, 3 hours, 6 hours, or 12 hours, inclusive). The concentrations of none or at least one of the nutrients of the fresh growth media can be brought to the concentrations of nutrients of the original growth media described in operation 102. The fresh growth media can have a volume that is greater than, less than, or equal to a volume of growth media that was lost from the original growth media during growth of the fungal cells in the original growth media.

In one example, after 6 hours, the concentration of sugar, phosphate-containing compound, and nitrogen-containing compound in the fresh growth media is increased. Nutrients are added to the broth to create a new broth. Nutrients are added to the broth to bring the concentrations of sugar, phosphate-containing compound, and nitrogen-containing compound of the new broth to the concentrations of sugar, phosphate-containing compound, and nitrogen-containing compound, respectively of the original growth media.

In one example, after at least 12 hours, 50% to 95% of the broth can be removed. Fresh media can be added containing nutrients (e.g., sugar, phosphate-containing compound, or nitrogen-containing compound). The nutrient concentration of the broth can be increased by adding fresh growth media.

Nutrients can be added in a continuous growth configuration. For example, a volume of broth (e.g., 0.01 vol %, 1 vol %, 5 vol %, 10 vol %, 25 vol %, 50 vol %, or 95 vol %, inclusive) can be removed from the container containing the fungal cells and the growth media. Fresh growth media can be added to the container containing the broth. The fresh growth media can be provided as a continuous flow. The volume of the broth in the container can be monitored to stay at a specified level. For example, the volume of the broth in the container can stay at a fixed volume. The volume of fresh growth media that is added can be equal to the volume of broth that is lost from the container.

The method 100 may include growing fungal cells in a growth media such that the fungal cells produce a mycelium mass having a protein content of greater than 40 wt % of a dry mass of the mycelium mass. For example, the mycelium mass can have a protein content of 45 wt %, 50 wt %, 60 wt %, 70 wt %, 80 wt %, or 90 wt %, inclusive, of the dry mass of the mycelium mass.

The method 100 includes separating the mycelium mass from the growth media, at 104. Separating the mycelium mass from the growth media can be performed using gravity straining, centrifugation, a belt press, a filter press, a mechanical press, a drum dryer, or any other suitable process. The separated mycelium mass can have a moisture content of greater than 90 wt %. For example, the separated mycelium mass can have a moisture content of 91 wt %, 92 wt %, 93 wt %, 94 wt %, 95 wt %, 96 wt %, 97 wt %, 98 wt %, or 99 wt %, inclusive. During the separation process, the mycelium mass can be washed with water, ethanol, acid, base or other solvent. Recovered filtrate can be reused or discarded. Cell walls of the mycelium mass can be disrupted, for example, through lysing. Lysis may be performed by adjusting the pH to below 4 or above 9, by adding lysis enzymes, by raising the temperature in a range of 40° C. to 60° C. in a range of 1 hour to 24 hours, or any other suitable lysis method. Following separation, additives (e.g., food additives) can be mixed with the mycelium mass. Additives can include vegetable or animal proteins, fats, emulsifiers, thickeners, stabilizers, and flavoring, for example, when the mycelium mass is being formed into an edible product.

The method 100 may include disposing the mycelium mass in a mold, at 106. Disposing the mycelium mass in a mold can include disposing the mycelium mass on a base of the mold, placing the mycelium mass or adding the mycelium mass to the mold. The mold can be of various shapes and sizes. For example, the mold can have sidewalls extending from the base. The sidewalls can hold the mycelium mass inside the mold. In some embodiments, the sidewalls of the mold are perforated. In some embodiments, the base of the mold can additionally or alternatively be perforated. For example, the base of the mold can have holes or perforations. In some embodiments, the mold is shaped as a chicken breast such that the compacted mycelium mass is shaped as a chicken breast. In other embodiments, the mold may be shaped as a chicken tender, a steak (e.g., a sirloin, a rib eye, a filet mignon, etc.) or any other suitable shape resembling an animal based meat product.

The method 100 may include applying uniaxial pressure to the mycelium mass to form a compacted mycelium mass, at 108. Applying uniaxial pressure to the mycelium mass may include applying pressure via a follower to produce a compacted mycelium mass. The follower can be of various shapes and sizes. For example, the follower may include a press or lid with a shape that fits into the mold. The follower can transfer the pressure to the mycelium mass to form the compacted mycelium mass having a moisture content in a range of 65 vol % to 85 vol %. For example, the compacted mycelium mass can have a moisture content of 65 wt %, 70 wt %, 75 wt %, 80 wt %, or 85 wt %, inclusive. The compacted mycelium mass can have a shape corresponding to a shape of the mold. Applying uniaxial pressure to the mycelium mass aligns mycelium fibers that form the mycelium mass in a plane orthogonal to the applied uniaxial pressure. Prior to applying uniaxial pressure on the mycelium mass, the mycelium fibers may be arranged in a random orientation throughout the mycelium mass. After uniaxial pressure is applied to the mycelium mass, the mycelium fibers are aligned in a plane orthogonal to the direction of the uniaxial pressure.

In some embodiments, the pressure of the uniaxial pressure is in a range of 25 psi to 300 psi. For example, the pressure can be 25 psi, 50 psi, 75 psi, 100 psi, 125 psi, 150 psi, 175 psi, 200 psi, 225 psi, 250 psi, 275 psi, or 300 psi, inclusive. In some embodiments, the mycelium mass is oriented in the mold in an x-y plane and the uniaxial pressure is applied in a z-direction. In some embodiments, the compacted mycelium mass has a hardness in a range of 0.007 kgf/mm² to 0.018 kgf/mm².

The method 100 may include slicing the compacted mycelium mass along a slicing plane, at 110. Slicing the compacted mycelium along a slicing plane can cause at least a portion of the plurality of fibers located at the slicing plane to be aligned in an orthogonal direction according to an orientation of slicing plane. By cutting through different planes of the block, substrate pieces can be formed with fibers aligned in specific directions at the cutting surface of the compacted mycelium based on the orientation of the slicing plane. For example, the slicing plane can be oriented at an angle of 0 degrees, 10 degrees, 20 degrees, 30 degrees, 40 degrees, 50 degrees, or 60 degrees, inclusive, relative to the x-y plane and the mycelium fibers included in the compacted mycelium at the cutting surface align parallel, 10 degrees, 20 degrees, 30 degrees, 40 degrees, 50 degrees, or 60 degrees, inclusive, off parallel to the respective slicing plane. In a food processing line, blades may be aligned vertically or horizontally. To achieve relative slicing planes, the compacted mycelium can be reoriented and then cut to integrate into conventional food processing equipment.

In some embodiments, the mycelium mass is oriented in the mold in an x-y plane and the uniaxial pressure is applied in a z-direction. In some embodiments, the slicing plane is oriented along the x-y plane causing the portion of the plurality of fibers to be parallel to the slicing plane and to have a chicken texture. In some embodiments, the slicing plane is oriented along a z-x plane causing the portion of the plurality of fibers to be orthogonal to the slicing plane and to have a beef texture. In some embodiments, the slicing plane is oriented at an angle from 0 degrees to 60 degrees along the x-y plane causing the portion of the plurality of fibers to be parallel to 0 degrees to 60 degrees off parallel and to have a fish texture

Following are some examples of growing fungi and obtaining a mycelium mass having a protein content of greater than 40 wt %. These examples are for illustrative purposes only and should not be construed as limiting the disclosure in any shape or form.

In one example, Neurospora crassa (N. crassa) was grown in batch configuration in a 10 L benchtop reactor. N. crassa is first grown on agar slants and incubated for 3 days at 32° C. Conidia or spores of the N. crassa are transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 2 g/L potassium phosphate monobasic, 1 g/L sodium nitrate, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 9.5 g/L. Protein analysis yields a crude protein content of 57 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. Conidia or spores of the N. crassa are transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 9 g/L. Protein analysis yields a crude protein content of 55 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 30 g/L sucrose, 3 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 11 g/L. Protein analysis yields a crude protein content of 63 wt %. Amino acid analysis yields a PDCAAS score of 1.0 for the fibrous mycelium mass. The fibrous mycelium mass has a combined methionine and cysteine content of 27 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 3.25 g/L urea, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, the mycelium is harvested using a cheese cloth, dewatered in a cider press, and completely dried in a dehydrator set at 74° C. The total cell dry weight is 8.5 g/L. Protein analysis yields a crude protein content of 56 wt %. Amino acid analysis yields a PDCAAS score of 1.0. The fibrous mycelium mass has a combined methionine and cysteine content of 25 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 15% ammonium hydroxide buffer. After 24 hours, the mycelium is harvested using a cheese cloth, dewatered in a cider press, and completely dried in a dehydrator set at 74° C. The total cell dry weight is 10 g/L. Protein analysis yields a crude protein content of 60 wt %. Amino acid analysis yields a PDCAAS score of 1.0. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 12 hours, 10 g/L sucrose and 1 g/L ammonium nitrate is added to the system. After 24 hours total, the mycelium is harvested using a cheese cloth, dewatered in a cider press, and completely dried in a dehydrator set at 74° C. The total cell dry weight is 12 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, 90% of the media is harvested; new media is added in the concentrations of above to bring the total system back to 10 L. The new sequential batch time is reduced to 12 hours. Every 12 hours 90% is harvested and the fed-batch process is repeated again. The process was carried out for 60 hours. The harvested cell dry weight is 9.5 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

In another example, N. crassa was grown in batch configuration in a 10 L benchtop reactor. N. crassa is first grown on agar slants and incubated for 3 days at 32° C. The conidia is transferred to a 250 mL vented Fernbach flask and grown for 48 hours on an orbital shaker table at 32° C. The resulting mycelium is aseptically transferred to a benchtop 10 L reactor containing the following media: 20 g/L sucrose, 2 g/L ammonium nitrate, 1 g/L potassium phosphate monobasic, 0.2 g/L magnesium sulfate, 0.1 g/L calcium chloride, and trace elements. Aeration is set at 0.75 vvm and agitation at 250 rpm. The pH is adjusted and held at 5.8 using a 6 N sodium hydroxide buffer. After 24 hours, 90% of the media is harvested; new media is added in the concentrations of above to bring the total system back to 10 L. The new sequential batch time is reduced to 12 hours. Every 12 hours 90% is harvested and the fed-batch process is repeated again. The process was carried out for 60 hours. Following straining with cheese cloth and pressing, all media is collected, autoclaved and reused by only adding 20 g/L sucrose, 2 g/L ammonium nitrate, and 1 g/L potassium phosphate monobasic. The repeated fed-batch process is carried out for 60 hours total. The harvested cell dry weight is 9.5 g/L. Protein analysis yields a crude protein content of 60 wt %. Amino acid analysis yields a PDCAAS score of 1.0 for the fibrous mycelium mass. The fibrous mycelium mass has a combined methionine and cysteine content of 26 mg/g crude protein.

A method of cell maintenance and isolation of conidia is described herein. Neurospora crassa (N. crassa) wild-type strain (FGSC #4815) was purchased from the fungal genetic stock center. The cells used for inoculations were stored on agar slants composed of 2% Vogel's 50x salts, 0.01% trace elements solution, 0.005% biotin, 1.5% sucrose, and 1.5% agar at −20° C. Growth experiments were started from cells removed from frozen agar slants onto new agar slants incubated at 30° C. for 2-3 days in complete darkness. Conidia were isolated from slants using standard methods and inoculated into 100 mL of fresh Vogel's medium (2% Vogel's 50x salts, 0.01% trace elements solution, 0.005% biotin, and 1.0% glucose) for batch submerged culture experiments. Conidial suspensions (1 mL in Vogel's medium) between optical densities of ˜0.7 were added to each culture.

A method of batch growth is described herein. Growth experiments were conducted in 1 L of fresh residual water. Batch cultures were incubated at 30° C. for 1-3 days (120 rpm) under constant light. Harvesting of biomass was performed using a vacuum filtration flask and then subsequently dried at 105° C.

The crude protein content of the filamentous fungus can be increased by supplementing with additional nitrogen sources. Non-limiting examples include supplementing with gaseous ammonia, liquid ammonia, ammonium nitrate, ammonium sulfate, sodium nitrate, yeast extract, urea, peptone, or other organic nitrogen source. A nitrogen source can be added with other pH buffering components. Non-limiting examples include acids, phosphates, borates, sulfates, and bases.

Following are some examples of growing fungi and forming directional mycelium fibers. These examples are for illustrative purposes only and should not be construed as limiting the disclosure in any shape or form.

In one example, Neurospora crassa (N. crassa) was grown in a 200 L stirred tank bioreactor under pre-set conditions. Following full growth, N. crassa biomass (mycelium) was collected through a 200 PEM nylon mesh bag. The moisture content of the mycelium at this stage was approximately 95%. The high moisture mycelium is added to a perforated, stainless-steel mold consisting of base and follower. The base has five fixed sides with perforation to allow for liquid to escape during compression. High moisture mycelium is needed to ensure no gaps in the block are formed. High moisture mycelium has the consistency of apple sauce or cheese curds and is somewhat fluid. A pressure of 100 psi is applied to the mold lid compacting the mycelium into a rigid block of approximately 75% moisture content. The block can be sliced in multiple directions to achieve fibers aligned in different directions.

In another example, Neurospora crassa (N. crassa) was grown in a 200 L stirred tank bioreactor under pre-set conditions. Following full growth, N. crassa biomass (mycelium) was collected through a 200 PEM nylon mesh bag. The moisture content of the mycelium at this stage was approximately 95%. The high moisture mycelium is added to a custom mold wherein either the base bottom or sides has a particular shape or the follower lid has a particular shape. A pressure of 100 psi is applied to the lid but now the final mycelium has a particular shape, such as a chicken breast, rather than a block. The new shapes still have fibers aligned in the direction of the plane perpendicular to the applied force.

The compacted mycelium mass can be used in a single or combination of ways. For example, the compacted mycelium mass can be cooked at a temperature of less than 100° C. (e.g., 90° C., 80° C., 75° C., or 50° C., inclusive) for 1-60 minutes in dry or steam environment. The compacted mycelium mass can be cooked at a temperature range of 100° C. to 200° C. (e.g., 100° C., 125° C., 150° C., or 200° C., inclusive) for 1-60 minutes in dry or steam environment. The compacted mycelium mass can be cooked in a water bath at less than 100° C. for 1 minute to 120 minutes (e.g., 1, 2, 5, 10, 20, 40, 60, 80, 100, or 120 minutes, inclusive).

In some embodiments, the compacted mycelium mass can be stored. The compacted mycelium mass can include additional ingredients. The compacted mycelium mass can be cooked. The compacted mycelium mass can be frozen at less than 0° C. under ambient or vacuum conditions, and/or refrigerated at less than 5° C. under ambient or vacuum conditions. The compacted mycelium mass can be stored indefinitely in sealed container.

Producing the compacted mycelium mass can include tuning the texture of the compacted mycelium mass. Texture of the compacted mycelium mass can be tuned by chemical washing of the mycelium mass. Alternatively, texture can be altered by controlling the water content of the mycelium mass. Texture can also be altered through the addition of different nutrients which determine mycelium mass growth and morphology. The density of final mycelium mass can be controlled by altering initial water content and drying conditions to produce a heavier or lighter end product.

Edible meat substitutes can be formed by dehydrating the compacted mycelium mass at temperatures less than 60° C. to achieve moisture contents less than 60%. The dehydrated mycelium mass can then be rehydrated in a marinade containing food additives, flavors, or colors. Depending on the initial slicing or pressing direction, different textures can be created corresponding to different meat substitutes such as a chicken substitute or beef substitute.

The edible meat substitute product can include a compacted mycelium mass in a range of 10 wt % to 100 wt % (e.g., 10 wt %, 20 wt %, 30 wt %, 40 wt %, 50 wt %, 60 wt %, 70 wt %, 80 wt %, or 100 wt %, inclusive). The edible meat substitute product can have a water content in a range of 0 wt % to 100 wt % (e.g., 0 wt %, 10 wt %, 20 wt %, 30 wt %, 40 wt %, 50 wt %, 60 wt %, 70 wt %, 80 wt %, or 100 wt %, inclusive). In some embodiments, the fibrous mycelium mass is in a range of 10 wt % to 50 wt %, and the water content is in a range of 50 wt % to 90 wt %. In some embodiments, the edible meat substitute product includes a soluble protein in a range of 1 wt % to 20 wt % (e.g., 1 wt %, 2 wt %, 5 wt %, 10 wt %, 15 wt %, or 20 wt %, inclusive). The edible meat substitute product can include a thickener content in a range of 0.01 wt to 5 wt % (e.g., 0.01 wt %, 0.05 wt %, 0.1 wt %, 1 wt %, 2 wt %, or 5 wt %, inclusive). The edible meat substitute product can include a fat source in a range of 0 wt % to 10 wt % (e.g., 0 wt %, 0.5 wt %, 1 wt %, 2 wt %, 5 wt %, or 10 wt %, inclusive).

The edible meat substitute product can include a flavorant. A flavorant can include flavorings or food additives. For example, the flavorant can include an oil, such as a nut-derived oil, vegetable-derived oil, plant-derived oil, and animal-derived oil. The flavorant can include spices (e.g., black pepper, fennel, mustard, nutmeg, cinnamon, ginger, cayenne pepper, clove, etc.). The flavorant can include a flavored powder (e.g., onion powder, garlic powder, BBQ powder, sour cream powder, lemon powder, lime powder, etc.).

The edible meat substitute product can include a combined methionine and cysteine content of at least 20 mg/gram crude protein. In some embodiments, the combined methionine and cysteine content in the edible meat substitute product is in a range of 20 mg/gram to 30 mg/gram (e.g., 20 mg/gram, 25 mg/gram, or 30 mg/gram, inclusive). The edible meat substitute product can have a PDCAAS score of 1. The edible meat substitute product can have an internal pH in a range of 2 to 9 (e.g., 2, 3, 4, 5, 6, 7, 8, or 9, inclusive). The edible meat substitute product can have a protein dry weight in a range of 20 wt % to 70 wt % (e.g., 20 wt %, 30 wt %, 40 wt %, 50 wt %, 60 wt %, or 70 wt %, inclusive). The edible meat substitute product can have a fiber dry weight in a range of 5 wt % to 30 wt % (e.g., 5 wt %, 10 wt %, 15 wt %, 20 wt %, 25 wt %, or 30 wt %, inclusive). The edible meat substitute product can have a dry fat weight of 0 wt % to 20 wt % (e.g., 0 wt %, 1 wt %, 5 wt %, 10 wt %, 15 wt %, or 20 wt %, inclusive). The edible meat substitute product can have a color represented by a CIE L* value of greater than 55. The chicken substitute product can have a hardness in a range of 0.00035 kgf/mm² to 0.018 kgf/mm², inclusive. The hardness can depend on whether the chicken substitute product is in a raw or cooked state.

The edible meat substitute product can include a chicken substitute product, a beef substitute product, a pork substitute product, a veal substitute product, or a fish substitute product. The edible meat substitute product can include 10 wt % to 90 wt % of the compacted mycelium mass (e.g., 10 wt %, 20 wt %, 30 wt %, 40 wt %, 50 wt %, 60 wt %, 70 wt %, 80 wt %, or 90 wt %, inclusive).

The chicken substitute product can include horizontally sliced fibers. The chicken substitute product can include 50 wt % to 90 wt % water (e.g., 50 wt %, 60 wt %, 70 wt %, 80 wt %, or 90 wt %, inclusive). The chicken substitute product can include 10 wt % to 50 wt % fungal mycelium such as from N. crassa (e.g., 10 wt %, 20 wt %, 30 wt %, 40 wt %, or 50 wt %, inclusive). The chicken substitute product can include 1 wt % to 20 wt % soluble protein (e.g., 1 wt %, 2 wt %, 5 wt %, 10 wt %, or 20 wt %, inclusive). The soluble protein can include pea, egg white, and potato, among others. The chicken substitute product can include 0.01 wt % to 5 wt % thickener (e.g., 0.01 wt %, 0.05 wt %, 0.1 wt %, 1 wt %, 2 wt %, or 5 wt %, inclusive). The thickener can include pectin, carrageenan, and agar, among others. The chicken substitute product can include 0 wt % to 10 wt % fat source (0 wt %, 1 wt %, 2 wt %, 3 wt %, 4 wt %, 5 wt %, or 10 wt %, inclusive). The fat source can include vegetable oils, seeds, among others. The chicken substitute product can include seasonings. The chicken substitute product can have various physical properties. For example, the chicken substitute product can have an internal pH in a range of 2 and 9 (e.g., 2, 3, 4, 5, 6, 7, 8, or 9, inclusive). The chicken substitute product can have a 40 wt % to 70 wt % protein dry weight (e.g., 40 wt %, 45 wt %, 50 wt %, 55 wt %, 60 wt %, 65 wt %, or 70 wt %). The chicken substitute product can have a 5 wt % to 30 wt % fiber dry weight (e.g., 5 wt %, 10 wt %, 15 wt %, 20 wt %, 25 wt %, or 30wt %, inclusive). The chicken substitute product can have a 0 w % to 10 wt % fat dry weight (0 wt %, 1 wt %, 2 wt %, 4 wt %, 5 wt %, or 10 wt %, inclusive). The chicken substitute product can have a CIE L* value greater than 55. The chicken substitute product can have a hardness in a range of 0.00035 kgf/mm² to 0.018 kgf/mm², inclusive. The hardness can depend on whether the chicken substitute product is in a raw or cooked state.

The beef substitute product can include fibers sliced at 45 degrees or termed a “bias cut”. The beef substitute product can include 50 wt % to 90 wt % water (e.g., 50 wt %, 60 wt %, 70 wt %, 80 wt %, or 90 wt %, inclusive). The beef substitute product can include 10 wt % to 50 wt % fungal mycelium such as from N. crassa (e.g., 10 wt %, 20 wt %, 30 wt %, 40 wt %, or 50 wt %, inclusive). The chicken substitute product can include 1 wt % to 20 wt % soluble protein (e.g., 1 wt %, 2 wt %, 5 wt %, 10 wt %, or 20 wt %, inclusive). The soluble protein can include pea, egg white, and potato, among others. The chicken substitute product can include 0.01 wt % to 5 wt % thickener (e.g., 0.01 wt %, 0.05 wt %, 0.1 wt %, 1 wt %, 2 wt %, or 5 wt %, inclusive). The thickener can include pectin, carrageenan, and agar, among others. The chicken substitute product can include 0 wt % to 10 wt % fat source (0 wt %, 1 wt %, 2 wt %, 3 wt %, 4 wt %, 5 wt %, or 10 wt %, inclusive). The fat source can include vegetable oils, seeds, among others. The beef substitute product can include seasonings. The beef substitute product can have various physical properties. For example, the beef substitute product can have an internal pH in a range of 2 and 9 (e.g., 2, 3, 4, 5, 6, 7, 8, or 9, inclusive). The beef substitute product can have a 40 wt % to 70 wt % protein dry weight (e.g., 40 wt %, 45 wt %, 50 wt %, 55 wt %, 60 wt %, 65 wt %, or 70 wt %). The beef substitute product can have a 5 wt % to 30 wt % fiber dry weight (e.g., 5 wt %, 10 wt %, 15 wt %, 20 wt %, 25 wt %, or 30wt %, inclusive). The beef substitute product can have a 0 wt % to 10 wt % fat dry weight (0 wt %, 1 wt %, 2 wt %, 4 wt %, 5 wt %, or 10 wt %, inclusive). The beef substitute product can have a hardness in a range of 0.00035 kgf/mm²to 0.011 kgf/mm², inclusive. The hardness can depend on whether the beef substitute product is in a raw or cooked state.

The meat substitute product can include 0 wt % to 90 wt % water (e.g., 0 wt %, 10 wt %, 20 wt %, 30 wt %, 40 wt %, 50 wt %, 60 wt %, 70 wt %, 80 wt %, or 90 wt %, inclusive). The meat substitute product can include 10 wt % to 100 wt % fungal mycelium such as from N. crassa (e.g., 10 wt %, 20 wt %, 30 wt %, 40 wt %, 50 wt %, 60 wt %, 70 wt %, 80 wt %, 90 wt %, or 100 wt %, inclusive). The meat substitute product can include 1 w % to 20 wt % soluble protein (e.g., 1 wt %, 2 wt %, 5 wt %, 10 wt %, or 20 wt %, inclusive). The soluble protein can include pea, egg white, and potato, among others. The meat substitute product can include 0 wt % to 5 wt % thickener (e.g., 0 wt %, 0.01 wt %, 0.05 wt %, 0.1 wt %, 1 wt %, 2 wt %, or 5 wt %, inclusive). The thickener can include pectin, carrageenan, and agar, among others. The meat substitute product can include 0 wt % to 50 wt % fat source (0 wt %, 10 wt %, 20 wt %, 30 wt %, 40 wt %, or 50 wt %, inclusive). The fat source can include vegetable oils, seeds, among others. The meat substitute product can include seasonings.

The compacted mycelium mass flavor can be enhanced by adding different oils. Non-limiting examples of oils include nut-derived, vegetable-derived, plant-derived, and animal-derived. Oils can be added to the food-grade residual water streams to have the multi-purpose use of acting as an antifoaming agent, a carbon source for the fungus, and to integrate extra/intracellularly into the mycelium mass. Alternatively, oil can be integrated into the mycelium mass following harvesting or following cooking.

Texture of the compacted mycelium mass can be tuned by chemical washing of the compacted mycelium mass. Alternatively, texture can be altered by controlling the water content of the compacted mycelium mass. Texture can also be altered through the addition of different nutrients which determine compacted mycelium mass growth and morphology. The density of final compacted mycelium mass can be controlled by altering initial water content and drying conditions to produce a heavier or lighter end product.

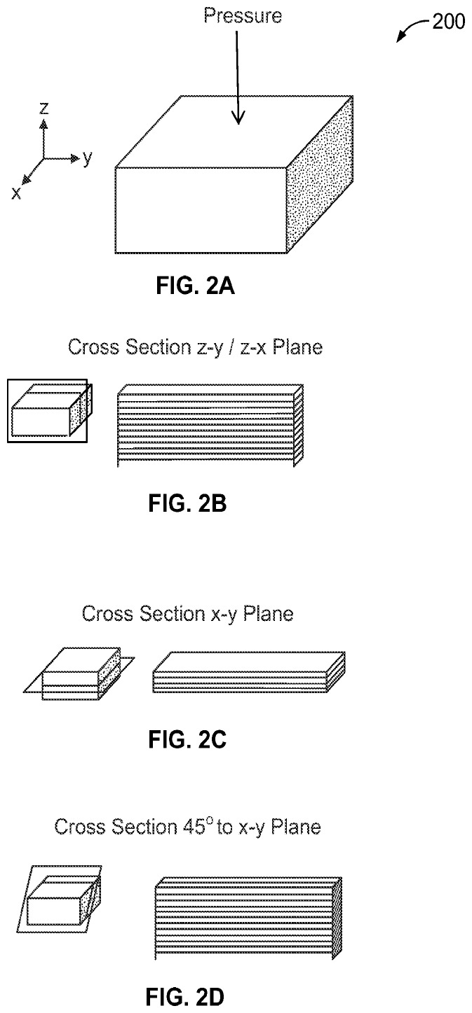

FIG. 2A is a perspective view of a mycelium block. In some embodiments, the mycelium mass is oriented in the mold in an x-y plane and the uniaxial pressure is applied in a z-direction. The pressure can be applied along the z-direction and the mycelium mass is oriented in the mold in the x-y plane. The mycelium block can be formed using uniaxial pressure and fixed boundaries. In some embodiments, the pressure of the uniaxial pressure is in a range of 90 psi to 110 psi. For example, the pressure can be 90 psi, 95 psi, 100 psi, 105 psi, or 110 psi, inclusive.

FIG. 2B illustrates a cross-sectional view of a mycelium block. FIG. 2B shows a cross-sectional slice of mycelium block in the z-y or z-x plane. In some embodiments, the slicing plane is oriented along a z-x plane causing the portion of the plurality of fibers to have a beef texture. In some embodiments, the slicing plane is oriented along a z-y plane causing the portion of the plurality of fibers to have a beef texture.

FIG. 2C illustrates a cross-sectional view of a mycelium block. FIG. 2C shows a cross-sectional slice of mycelium block in the x-y plane. In some embodiments, the slicing plane is oriented along the x-y plane causing the portion of the plurality of fibers to have a chicken texture.

FIG. 2D illustrates a cross-sectional view of a mycelium block. FIG. 2D shows a cross-sectional slice of mycelium block at an offset to the x-y plane. In some embodiments, the slicing plane is oriented at an angle from 0 degrees to 60 degrees along the x-y plane causing the portion of the plurality of fibers to have a fish texture. For example, the slicing plane can be oriented at an angle of 0 degrees, 10 degrees, 20 degrees, 30 degrees, 40 degrees, 50 degrees, or 60 degrees, inclusive, along the x-y plane.

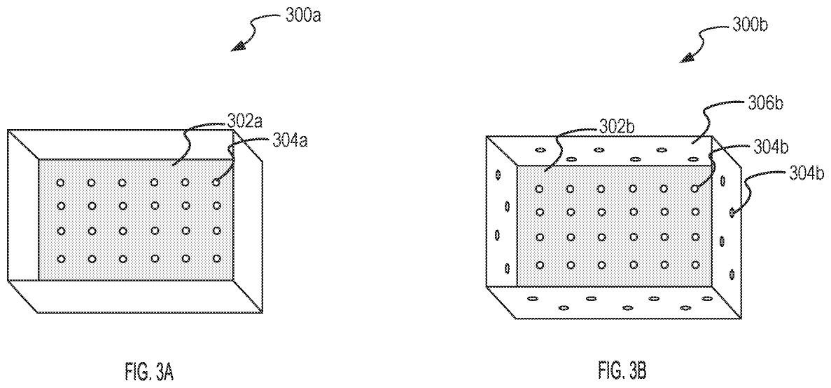

FIG. 3A is a perspective view of a mold 300a, according to an embodiment. The mold 300a includes a base 302a that defines a plurality of perforations 304a (e.g., holes, slits, gaps, openings, etc.) therethrough. The perforations 304a can be arranged in a pattern (e.g., regularly spaced, periodic, randomly spaced, etc.). The perforations 304a can allow moisture to be separated from the mycelium mass. The perforations 304a can allow moisture to be separated from the mycelium mass when the uniaxial pressure is applied. The mold 300a can be made of stainless steel.

FIG. 3B illustrates a perspective view of a mold 300b, according to another embodiment. The mold 300b includes a base 302b and sidewalls 306b, each of which define a plurality of perforations 304b (e.g., holes, slits, gaps, openings, etc.) therethrough. The perforations 304b can be arranged in a pattern (e.g., regularly spaced, periodic, randomly spaced, etc.). The perforations 304b can allow moisture to be separated from the mycelium mass. The perforations 304b can allow moisture to be separated from the mycelium mass when the uniaxial pressure is applied. The mold 300b can be made of stainless steel. The base 302b and the sidewalls 306b can be made of different materials.

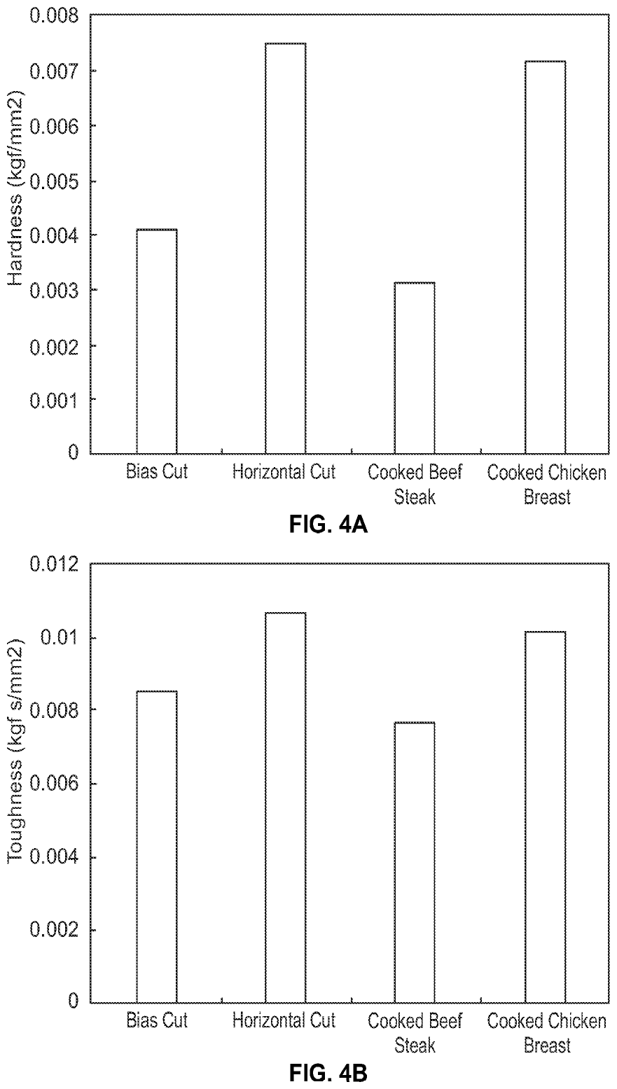

FIG. 4A illustrates a bar chart of hardness values for compacted mycelium that has been cut in different orientations. FIG. 4B illustrates a bar chart of toughness values for compacted mycelium that has been cut in different orientations are corresponding to different cuts of meat. The compacted mycelium can include a compacted, sliced, dehydrated and rehydrated mycelium mass. The compacted mycelium can have a bias cut. A bias cut can include a slicing plane that is inclined at an angle of 45 degrees from the x-y plane. The compacted mycelium can have a horizontal cut. A horizontal cut can include the plurality of fibers in the x-y plane. The bias cut can match closely to a cooked beef steak hardness and toughness while a horizontal cut correlates in hardness and toughness to a cooked chicken breast.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A method of forming an edible meat substitute product, comprising:
growing fungal cells in a growth media such that the fungal cells produce a mycelium mass having a protein content of greater than 40 wt % of a dry mass of the mycelium mass;
separating the mycelium mass from the growth media;
disposing the mycelium mass on a base of a mold, the mold having sidewalls extending from the base, at least the base of the mold being perforated; and
applying a uniaxial pressure to the mycelium mass via a follower to produce a compacted mycelium mass having a moisture content in a range of 65 vol % to 85 vol % and having a shape corresponding to a shape of the mold,
wherein a plurality of fibers of the compacted mycelium mass are aligned in a direction orthogonal to the direction of the applied uniaxial pressure.

2. The method of claim 1, wherein the sidewalls of the mold are perforated.

3. The method of claim 1, wherein the mold is shaped as a chicken breast such that the compacted mycelium mass is shaped as a chicken breast.

4. The method of claim 1, wherein the uniaxial pressure is in a range of 25 psi to 300 psi.

5. The method of claim 1, wherein the mycelium mass is oriented in the mold in an x-y plane and the uniaxial pressure is applied in a z-direction.

6. The method of claim 5, further comprising:
slicing the compacted mycelium mass along a slicing plane causing at least a portion of the plurality of fibers located at the slicing plane to be aligned in an orthogonal direction according to an orientation of the slicing plane.

7. The method of claim 6, wherein the slicing plane is oriented along the x-y plane causing the portion of the plurality of fibers to be parallel to the slicing plane and to have a chicken texture.

8. The method of claim 6, wherein the slicing plane is oriented along a z-x plane causing the portion of the plurality of fibers to be orthogonal to the slicing plane and have a beef texture.

9. The method of claim 6, wherein the slicing plane is oriented at an angle from 0 degrees to 60 degrees along the x-y plane causing the portion of the plurality of fibers to be parallel to 0 to 60 degrees off parallel and to have a fish texture.

10. The method of claim 1, wherein the compacted mycelium mass has a hardness in a range of 0.00035 kgf/mm² to 0.018 kgf/mm².

11. The method of claim 1, wherein separating the mycelium mass from the growth media produces a separated mycelium mass, the separated mycelium mass having a moisture content of greater than 90 wt %.

12. A method of forming an edible meat substitute product, comprising:
providing a mycelium mass having a protein content of greater than 40 wt % of a dry mass of the mycelium mass;
disposing the mycelium mass on a mold; and
applying a uniaxial pressure to the mycelium mass via a follower to produce a compacted mycelium mass having a moisture content in a range of 65 vol % to 85 vol % and having a shape corresponding to a shape of the mold,
wherein a plurality of fibers of the compacted mycelium mass are aligned in a direction orthogonal to the direction of the applied uniaxial pressure.

13. The method of claim 12, wherein the mold is shaped as a chicken breast such that the compacted mycelium mass is shaped as a chicken breast.

14. The method of claim 12, wherein the uniaxial pressure is in a range of 25 psi to 300 psi.

15. The method of claim 12, wherein the mycelium mass is oriented in the mold in an x-y plane and the uniaxial pressure is applied in a z-direction.

16. The method of claim 15, further comprising:
slicing the compacted mycelium mass along a slicing plane causing at least a portion of the plurality of fibers located at the slicing plane to be aligned in an orthogonal direction according to an orientation of the slicing plane.

17. The method of claim 16, wherein the slicing plane is oriented along the x-y plane causing the portion of the plurality of fibers to be parallel to the slicing plane and to have a chicken texture.

18. The method of claim 16, wherein the slicing plane is oriented along a z-x plane causing the portion of the plurality of fibers to be orthogonal to the slicing plane and have a beef texture.

19. The method of claim 16, wherein the slicing plane is oriented at an angle from 0 degrees to 60 degrees along the x-y plane causing the portion of the plurality of fibers to be parallel to 0 to 60 degrees off parallel and to have a fish texture.

20. The method of claim 12, wherein the compacted mycelium mass has a hardness in a range of 0.00035 kgf/mm²to 0.018 kgf/mm².
